(19) United States
(12) Patent Application Publication
(10) Pub. No.: US 2005/0203063 A1
(43) Pub. Date: Sep. 15, 2005

(54) Proteasome pathway inhibitors and related methods

(76) Inventors: Raymond Deshaies, Claremont, CA (US); Randall King, Boston, MA (US); Rati Verma, Altadena, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/940,502

(22) Filed: Sep. 13, 2004

Related U.S. Application Data
(60) Provisional application No. 60/502,540, filed on Sep. 12, 2003.

(51) Int. Cl. A61K 31/695; A61K 31/655; A61K 31/44; A61K 31/40; A61K 31/277; A61K 31/195

(52) U.S. Cl. 514/63; 514/561; 514/408; 514/521; 514/357; 514/151; 514/150

(57) ABSTRACT

The disclosure provides compositions and methods for blocking the proteasome pathway, as well as compounds that block mitotic cell cycle progression. Compounds disclosed include a family of molecules that bind to a multiubiquitin chain attached to a protein and thereby inhibit degradation of that protein by the proteasome pathway. According to another aspect of the disclosure, compounds are provided that inhibit cell cycle progression. Compounds disclosed herein may be formulated for pharmaceutical use and employed in methods for treating cancers or other hyperproliferative disorders.

### RELATED APPLICATIONS

This application claims the benefit of U.S. Provisional Application Ser. No. 60/502,540 filed Sep. 12, 2003 and entitled “Method and Compositions for Modulating Proteasome Pathway Inhibition”. The above-referenced provisional application is incorporated by reference herein.

### STATEMENT REGARDING FEDERAL FUNDING

Work described herein was funded, in part, by National Institutes of Health Grant Nos. NCI P01 CA78048 and NIGMS RO1 GM66492. The United States government has certain rights in the invention.

### BACKGROUND

Protein turnover in cells is regulated in part by the ubiquitin-dependent proteasome pathway. Ubiquitin (Ub) is involved in the post-translational regulation and degradation of a multitude of proteins. The covalent attachment of multiubiquitin modification on a protein will generally target that protein for degradation by a cellular proteasome. Ubiquitin molecules can be linked to each other through different internal lysines, including K29, K48 and K63. The K48-linked form of multiubiquitin is the principle targeting signal for proteolysis. K63-linked chains are implicated in enzyme regulation; no physiological function has been identified for K29-linked chains.

Ubiquitin is added to polypeptides by a post-translational process in which ubiquitin chains or single ubiquitin molecules are appended to target proteins, giving rise to multi- or monoubiquitination. Conjugation of Ub onto proteins generally involves the concerted activity of three enzymes: a ubiquitin-activating enzyme (E1), a ubiquitin-conjugating enzyme (E2), and ubiquitin ligase (E3). Multiubiquitinated proteins (with a K48-linked Ub chain) are then targeted for degradation by the proteasome.

Inhibitors of the proteosome pathway are known to have anticancer effects. It is also known that the mitotic cell cycle is driven by a series of regulated proteasome-dependent protein degradation events. A compound that inhibits the proteasome pathway, bortezomib, [(1R)-3-methyl-1-[[(2S)-1-oxo-3-phenyl-2-[(pyrazinylcarbonyl)amino]propyl]amino]butyl] boronic acid, was approved in May 2003 by the U.S. Food and Drug Administration for the treatment of relapsed multiple myeloma and is sold under the trade name VELCADE. Bortezomib inhibits the proteasome pathway by inhibiting protease activity within the 20S proteasome. The bortezomib approval establishes that the proteasome pathway is an important and useful therapeutic target for developing anticancer therapeutics.

Because of the prevalence and severity of many cancers, there is a clear need for improved therapeutic approaches for treating cancers. In addition, regulators of the cell cycle generally are expected to have anticancer effects, and may affect the proteasome-dependent protein degradation pathway directly or indirectly.

Certain compounds described herein have been suggested to have anti-viral properties, such compounds are described in U.S. Pat. No. 5,681,832.

There is a need for new methods to discover compounds that interact with, and directly or indirectly regulate, the ubiquitin dependent proteasome pathway.

### SUMMARY

The disclosure provides compositions and methods for blocking the proteasome pathway, as well as compounds that block mitotic cell cycle progression. According to one aspect of the disclosure, a compound is provided that binds to the multiubiquitin chain attached to a protein and thereby inhibits degradation of that protein by the proteasome pathway. According to another aspect of the disclosure, a compound is provided that inhibits cell cycle progression. Compounds disclosed herein may be formulated for pharmaceutical use and employed in methods for treating cancers or other hyperproliferative disorders.

According to certain methods disclosed herein, a cell or organism is treated with a compound that interferes with a ubiquitinated protein's entry into or processing within the proteasome pathway, but upstream of the final proteolysis step that occurs in the 20S proteasome. According to one embodiment, the compound binds to a K48-linked multiubiquitin chain attached to a protein that is to be degraded. The multiubiquitin chain is a signal for degradation of the protein to which it is attached, and is later removed from the targeted protein by the action of the proteasomal isopeptidase Rpn11. In a second embodiment of the invention, the compound blocks a step between the binding of a substrate's multiubiquitin chain signal to the proteasome, and the subsequent removal of the multiubiquitin chain from the substrate by the isopeptidase activity of Rpn11.

The disclosed methods include a screening method to identify compounds that act on the ubiquitin-dependent proteasome pathway, but do not inhibit the ultimate proteolytic steps that occur within the 20S proteasome. According to certain methods of the disclosure, a compound is tested for its ability to inhibit accumulation of a deubiquitinated protein in the presence of 26S proteasome. According to another method of the disclosure, a compound is tested for its ability to inhibit disappearance of ubiquitinated substrate in the presence of 26S proteasome in the presence of an inhibitor of one or more 20S proteasomal proteases. Suitable proteasome inhibitors include VELCADE, Epoxomicin, and YU-101 (Elofsson, M., Splittgerber, U., Myung, J., Mohan, R. & Crews, C. M. (1999) Chem. Biol. 6, 811-822).

The disclosure also provides methods for identifying compounds that bind to the multiubiquitin chain. According to such methods, a candidate compound is contacted with a lysine-48-linked multiubiquitin chain and binding on the compound to the multiubiquitin chain is detected.

The disclosure also provides a method of treating a patient suffering from a disease which can be treated by administering a proteasome inhibitor. One such method involves administering to a patient a compound that inhibits the proteasome pathway after a target protein has been ubiquitinated, but prior to the ultimate degradation step by the 26S proteasome. Another method involves administering to a patient a therapeutically effective combination of a proteasome protease inhibitor (e.g. VELCADE) along with a compound that inhibits the proteasome pathway after a target protein has been ubiquitinated, but prior to the ultimate degradation step by the 26S proteasome.

### BRIEF DESCRIPTION OF THE DRAWINGS

FIG. 1. Functional classification of inhibitors. Compounds that scored in the initial screen were reacquired as dry powders and retested in Xenopus extracts in 4 assays at 200 μM concentration (32) as described (6). Data is shown for the twenty-two most active compounds whose chemical structures were confirmed. (A) Compounds and cyclin-luciferase were added to interphase extracts, which were then induced to enter mitosis by addition of non-degradable cyclin B. Percent inhibition of cyc-luc turnover by each compound is coded according to the grayscale shown at the bottom of the figure; specific values are available in Table 1. (B) Extracts were pre-treated with non-degradable cyclin B to allow entry into mitosis prior to addition of test compound and reporter protein. (C) Interphase extracts were treated by addition of recombinant Cdh1 to activate cyc-luc turnover. (D) Interphase extracts were treated with recombinant axin to induce turnover of β-catenin-luciferase.

FIG. 2. Class IIB compounds inhibit degradation and deubiquitination of UbSic1 by purified 26S proteasomes by binding to K48-linked multiubiquitin chains. (A) Degradation of UbSic1. Purified 26S proteasomes were preincubated in the presence or absence of test compounds. UbSic1 was then added, and degradation assayed as described (6). Py mock refers to the pyridine solvent in which C23 was dissolved which was marginally inhibitory, unlike the 50% DMSO vehicle used for other test compounds. (B) Deubiquitination of UbSic1. Purified 26S proteasomes were pre-incubated with 100 μM epoxomicin in the presence or absence of 100 μM test compound. UbSic1 was then added, and deubiquitination was assayed as described (6). (C) Titration curve with varying concentrations of C92 to determine half-maximal inhibition of deubiquitination. (D) C92 inhibits binding of UbSic1 to 26S proteasomes. Purified 26S proteasomes immobilized on anti-Flag beads were incubated with UbSic1 in the presence or absence of C92 as described (6). Following binding, beads were washed and analyzed by immunoblotting with anti-Sic1 polyclonal serum. (E) Binding of UbSic1 to the polyUb binding proteins Rpn10 and Rad23 is inhibited by C92. Recombinant Gst-Rpn10 and Gst-Rad23 (1 μg each) were immobilized on glutathione sepharose beads and then incubated with UbSic1 in the presence or absence of C92. Following binding, beads were washed and analyzed by immunoblotting. (F) C92 interacts with multiUb chains. Equivalent amounts (5 μg) of purified Gst, Gst-fusion protein or multiUb chains were incubated with C92 or C1 before being loaded on a 5% native polyacryamide gel. Electrophoresis was performed as described (33). (G) C92 and C59 interact specifically with K48-linked Ub on native gels. Ub (16 μM), K48-linked diUb (8 μM), or tetraUb chains (8 μM) were preincubated with a two fold-molar excess (monoUb and diUb), or equivalent amounts (tetraUb) of test compounds before being resolved on native gels as in (G). Tetra K29Ub, K48Ub, and K63Ub refer to tetraubiquitin chains wherein the ubiquitins are linked via K29, K48, or K63.

FIG. 3. Ubistatin A binding to K48-linked di-ubiquitin induces site-specific perturbations in NMR spectra for both Ub domains. (A) Backbone NH chemical shift perturbation, Δδ, and percent signal attenuation caused by ubistatin A binding as a function of residue number for the distal (left) and the proximal (right) domains. Ub units are called “distal” and “proximal” to reflect their location in the chain relative to the free C-terminus. The diagram (top) depicts the location of the G76-K48 isopeptide bond between the two Ub domains. Residues T7 in both domains and R72 in the distal Ub (marked with the stars) show significant signal attenuation that could not be accurately quantified due to signal overlap. (B) Mapping of the perturbed sites on the surface of di-Ub. The distal and proximal domains are shown in surface representation colored blue and green, respectively; the perturbed sites on these domains are colored yellow and red and correspond to residues with Δδ>0.075 ppm and/or signal attenuation greater than 50%. Numbers indicate surface location of the hydrophobic-patch and some basic residues, along with G76 (distal) and the side chain of K48 (proximal). The atom coordinates are from the crystal structure 1AAR.pdb (26).

FIG. 4. Ubistatin A blocks protein turnover in animal cells. HEK-293 cells stably expressing GFP-AR were microinjected with Protac and rhodamine dextran in the absence (A) or presence of 100 nM ubistatin A (B) or 100 nM epoxomicin (C). The results are representative of experiments performed on 3 separate days.

FIG. 5. (A) Complete inhibition of deubiquitination by 100 μM C59, and marginal inhibition by 100 μM C23. Assays were performed and evaluated as summarized in the legend to FIG. 2B. The metal chelating reagent phenanthroline is designated as “P”, and “Py” refers to the pyridine solvent that is used as the solvent for C23. (B) Titration curve for C59. (C) Inhibition of binding of ubiquitin conjugates to Gst-Rpn10 by C92 and C59, but not other compounds. The experiment was performed as described for FIG. 2E.

FIG. 6. Class IIB compounds do not compromise 26S proteasome integrity at concentrations that inhibit ubiquitin-dependent proteolysis. (A) Purified 26S proteasomes were preincubated with test compounds before being resolved on a native gel. Following electrophoresis, the gel was stained with Coomassie Blue. (B) C92 does not affect 20S core activity. Same as (A), except that following electrophoresis, the gel was incubated with fluorescent substrate to determine peptidase activity, which was visualized using a UV transilluminator (9). C23 was also without effect (not shown), whereas C59 was too fluorescent by itself for this assay. (C) Untreated 26S proteasomes were resolved on a native gel. Following electrophoresis, the gel was incubated with the peptidase substrate in the presence of ubistatin. No inhibition could be observed, whereas epoxomicin inhibited peptidase activity under identical conditions (not shown).

FIG. 7. Synergy between conventional proteasome inhibitors and C92. The figure shows the effects of compound 92 (C92), MG132, or C92 plus MG132 (a conventional proteasome inhibitor) on the turnover of a cyclin-luciferase reporter in Xenopus extracts. This is the same assay as described in FIG. 1. The combination of C92 plus MG132 (upper trace) show synergistic inhibition of cyclin proteolysis in extracts.

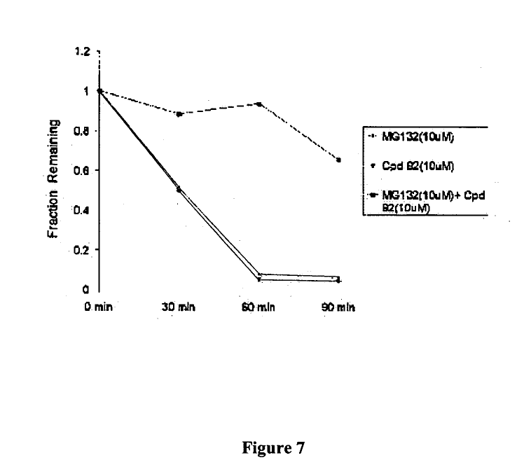

### DETAILED DESCRIPTION

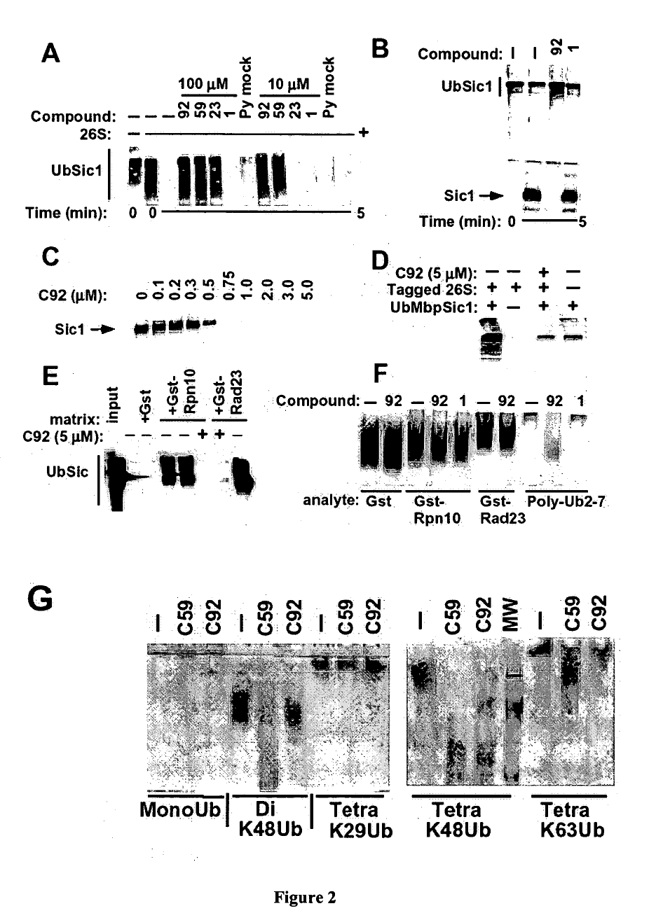

In certain aspects the disclosure provides methods that comprise treating a cell or organism with a compound that interferes with a ubiquitinated protein's entry into or processing within the proteasome pathway, but upstream of the final proteolysis step that occurs in the 20S proteasome. Compounds belonging to a novel class of pharmacological agents, referred to herein as “ubistatins”, may be used in such a method. A ubistatin is a compound that binds to K48-linked multiubiquitin chains and inhibits the degradation of a protein that is covalently attached to such a multiubiquitin chain. Preferably, a ubistatin will bind to a K48-linked (i.e. G76-K48 linked) multiubiquitin chain with greater affinity than to a K29 or K63-linked multiubiquitin chain. A ubistatin will preferably cause half-maximal inhibition of proteasome-mediated degradation in an in vitro assay (such as that described in FIG. 2B and the Examples) at a concentration of about 500 nM or less and will preferably cause substantial inhibition of the binding of a K48-linked multiubiquitin chain to one or more multiubiquitin binding proteins, such as Rpn10 or Rad23, or homologs thereof, at concentrations of 5 μM or less. Binding assays may be performed as described in FIG. 2E and the Examples. As suggested herein, a ubistatin may be designed so as to bind to a patch of residues on the Ub-Ub interface of the K48-linked chain, including any or all of L8, I44, V70, K6, K11, R42, H68 and R72. Two examples of ubistatins are referred to herein as compound 59 (C59) and compound 92 (C92), or ubistatin A and ubistatin B, respectively. These compounds have the following structures: 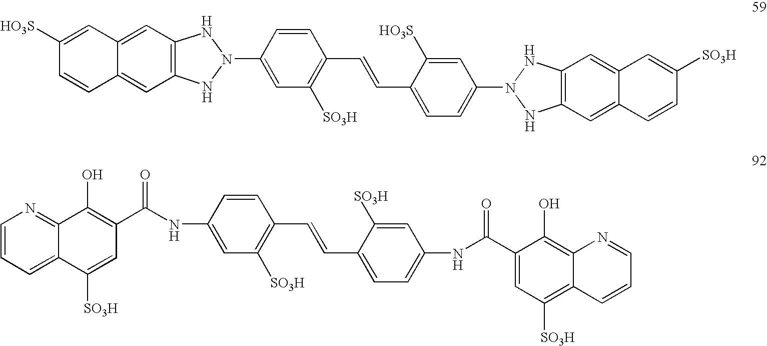
Furthermore, elimination of one or more of the sulfates tends to attenuate the inhibitory effect on the proteasome pathway. Accordingly, a ubistatin may be designed so as to have two, three, four or more negative charges at physiological pH, arranged along a relatively hydrophobic core. Ubistatins disclosed herein may be designed so as to have a structural framework according to Formula (I) below
A¹-L¹-B¹-M-B²-L²-A² (I)
or a pharmaceutically acceptable salt or prodrug thereof, wherein as valence and stability permits:
L¹, L², and M independently for each occurrence, represent a direct bond, —(CR¹R²)ₙ—, —(CR¹═CR²)ₙ—, —O—, —S—, —Se—, —NR³—, —(N═N)ₙ—, —O—N═CH—, —(R₃)N—N(R₃)—, —O—N(R₃)—, —NR³—C(O)—, —C(O)—NR³—, —C(O)—, —C(S)—, —O—C(O)—O—, —O—C(S)—O—, —NR³—C(O)—NR³—, —O—C(O)—, —C(O)—O—, —NR³—C(S)—NR³—, —O—C(S)—, —C(S)—O—, —S(O)ₘ—, or —C(O)—C(O)—; and
A¹, A², B¹ and B² independently for each occurrence, are absent or represent aryl, heteroaryl, cycloalkyl, cycloalkenyl, heterocyclyl, aralkyl, heteroaralkyl, or polycyclyl, with 1-3 substitutions of R^{a} and 0-3 substitutions of R^{b};
wherein any one of A¹, A², B¹ and B² are optionally connected to any one of the others by one or more bonds;
wherein R^{a}, independently for each occurrence, represents —S(O)ₘ—OH, —CO₂H, —P(O)—(OH)ₘ, —OH, or a moiety having an ionizable hydrogen and a pKa of less than 10;
R^{b}, independently for each occurrence, represents one or more of hydrogen, halogen, hydroxyl, alkoxyl, silyloxyl, amino, nitro, sulfhydryl, alkylthio, imino, amido, cyano, carbonyl, carboxyl, silyl, sulfamoyl, sulfinyl, thioalkyl, alkylsulfonyl, arylsulfonyl, acyl, formyl, esteryl, isocyano, guanidinyl, amidinyl, acetalyl, ketalyl, amine oxidyl, azido, carbamyl, hydroxamyl, imidyl, oximyl, sulfonamidyl, thioamidyl, thiocarbonyl, ureayl, thioureayl, or a substituted or unsubstituted alkyl or, alkenyl, alkynyl, aryl or heteroaryl or —(CH₂)ₙ—R₄;
R¹, R², R³, independently for each occurrence, represent hydrogen, alkoxy, halogen, lower alkyl (substituted or unsubstituted), aryl (substituted or unsubstituted), aralkyl (substituted or unsubstituted), heteroaryl (substituted or unsubstituted), or heteroaralkyl (substituted or unsubstituted);
R₄, independently for each occurrence, represents a substituted or unsubstituted aryl, cycloalkyl, cycloalkenyl, heterocyclyl, heteroaryl, or polycyclyl;
m, independently for each occurrence, represents an integer from 1 to 2; and
n, independently for each occurrence, represents an integer from 0 to 3.

Optionally, M is —(CR¹═CR²)ₙ—, —(N═N)ₙ—, or —(R₃)N—N(R₃)—, and n is 1. Optionally, B¹ and B² is aryl or heteroaryl. Optionally, L¹ and L² is a direct bond. Optionally, L¹ and L² is —NR³—C(O)— or —C(O)—NR³—. Optionally, A¹ and A² is aryl, heteroaryl, or polycyclyl. Optionally, one or more R^{a} is —S(O)ₘ—OH and m is 2. Optionally, M is —(CR¹═CR²)ₙ—, wherein n is 1 and both R¹ and R² are hydrogen. Optionally, B¹ and B² are phenyl. Optionally, A¹ and A² are naphthyl. Optionally, A¹ and A² are naphthotriazole. Optionally, each instance of A¹, A², B¹ and B² is substituted by at least one R^{a}, wherein R^{a} is —S(O)ₘ—OH and wherein m is 2.

In addition to the ubistatins, the disclosure provides a large number of compounds that are effective to inhibit cell cycle progression. For example, the compounds of groups IA and IB, below, are effective to block entry into mitosis and/or activation of APC/C. Accordingly, any of these compounds and structurally similar variants may be used to inhibit proliferation of, for example, a cancer cell. 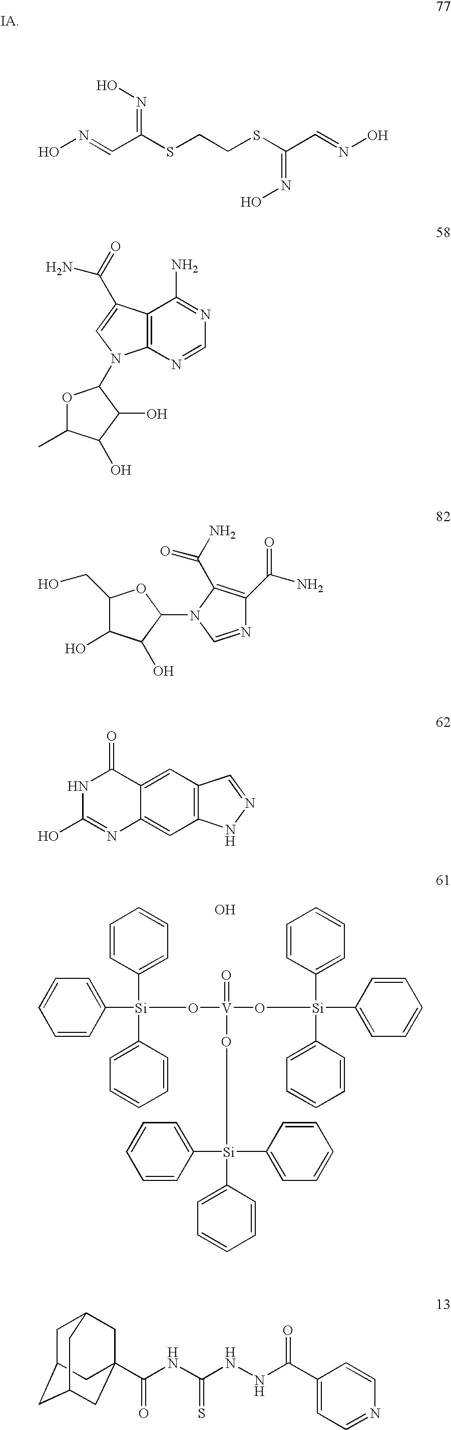 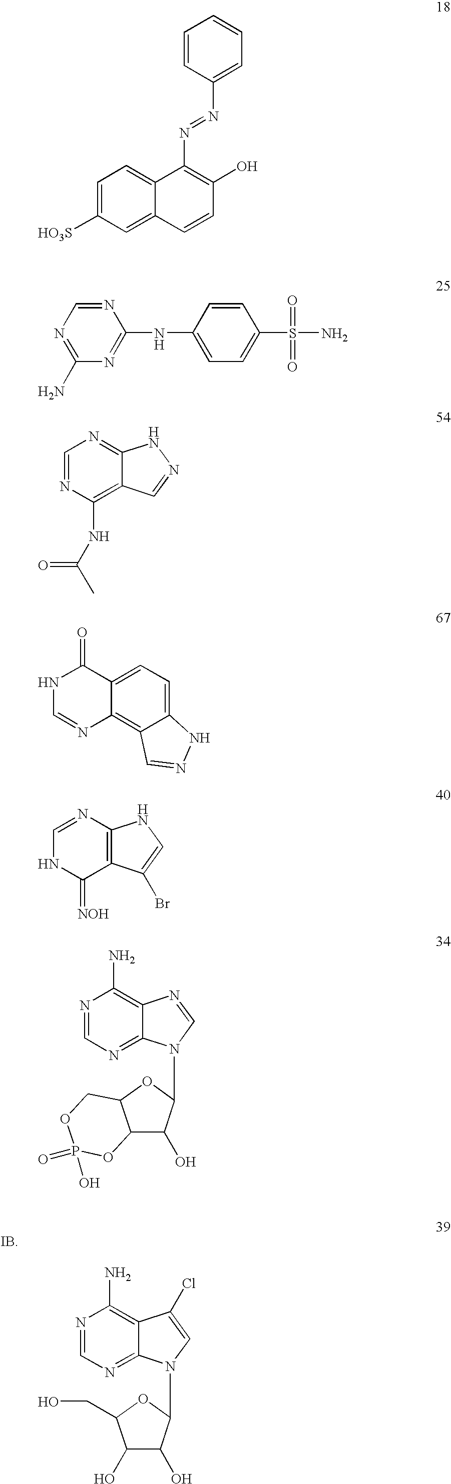 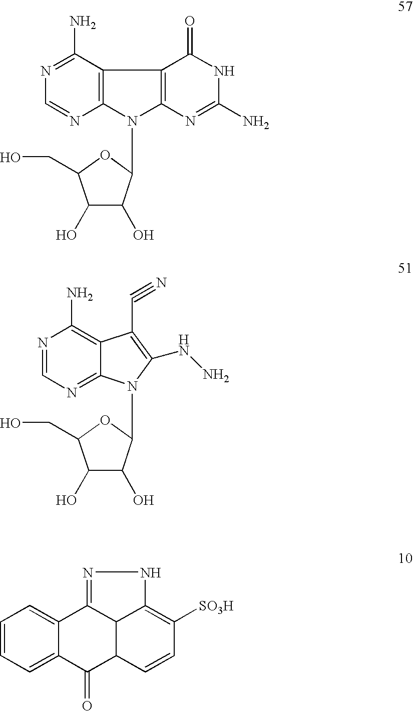
Likewise, compounds of the group IIA block the function of an activated APC/C or act downstream, such as on the proteasome itself. Compounds of this group, and structurally related variants, likewise inhibit cell cycle progression and may therefore be used to inhibit cellular proliferation.

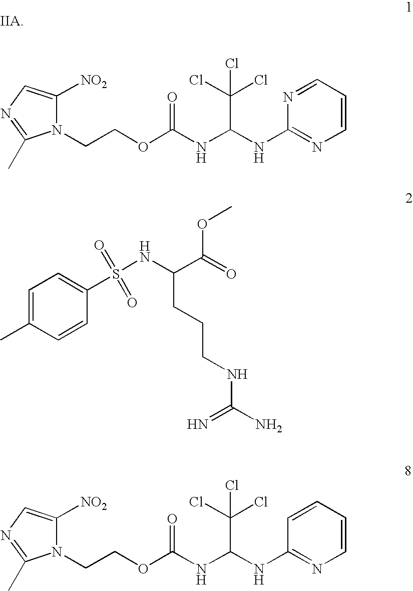

An additional compound with cell cycle inhibitory activity is compound 94:

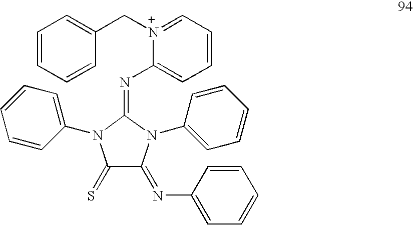

The numbered compounds described herein are publicly available, either through the Chembridge Corporation (San Diego, Calif.; product name Diverset E) or the National Cancer Institute Diversity set from the NCI open collection. A table of the compounds is presented below.

TABLE 1

| Cmpd | Class | A | B | C | D | ID # | CAS |
|---|---|---|---|---|---|---|---|
| 77 | IA | 100 | 4 | −12 | 0 | NSC383123 | NA |
| 58 | IA | 100 | 5 | −8 | 2 | NSC298884 | 65562-56-3 |
| 82 | IA | 100 | 0 | 0 | 0 | NSC519257 | 66678-51-1 |
| 62 | IA | 84 | 0 | −8 | 0 | NSC350006 | 73908-01-7 |
| 61 | IA | 77 | 8 | −8 | 2 | NSC349960 | 18822-50-9 |
| 13 | IA | 75 | 0 | −9 | 0 | C5144324 | 301860-02-6 |
| 18 | IA | 73 | 4 | −7 | 0 | NSC7831 | 1934-20-9 |
| 25 | IA | 66 | 3 | −6 | 0 | NSC19742 | 92474-98-1 |
| 54 | IA | 54 | 3 | −6 | 0 | NSC205359 | 68341-64-0 |
| 67 | IA | 53 | 3 | −8 | 3 | NSC350138 | 81115-64-2 |
| 40 | IA | 42 | 0 | −6 | 3 | NSC124151 | 22276-98-8 |
| 34 | IA | 100 | 0 | −8 | 6 | NSC94017 | 60-92-4 |
| 39 | IB | 100 | 9 | −7 | 67 | NSC124149 | 24386-95-6 |
| 57 | IB | 100 | 4 | 0 | 60 | NSC279846 | 73024-72-3 |
| 51 | IB | 100 | 0 | 0 | 30 | NSC172599 | 22256-94-6 |
| 10 | IB | 33 | 0 | −4 | 21 | C5255908 | NA |
| 1 | IIA | 100 | 100 | 35 | 6 | C5271852 | NA |
| 2 | IIA | 80 | 50 | 100 | 0 | C5117023 | 901-47-3 |
| 8 | IIA | 70 | 63 | 20 | 0 | C145663 | NA |
| 23 | IIB | 100 | 100 | 100 | 27 | NSC14226 | 5429-79-8 |
| 59 | IIB | 97 | 100 | 100 | 70 | NSC306455 | NA |
| 92 | IIB | 60 | 22 | 65 | 21 | NSC665534 | NA |

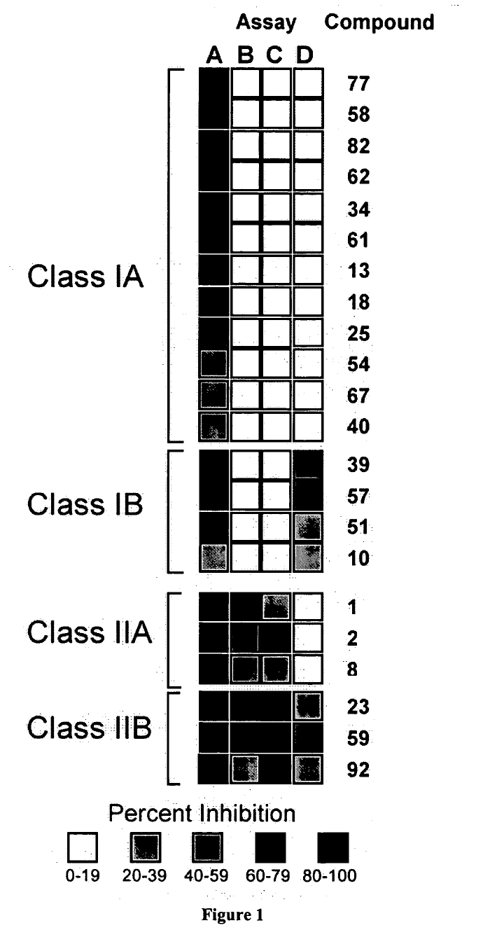

Table 1: Activity and identification numbers for compounds reported in FIG. 1. The “Cmpd” column corresponds to the numbers used in FIG. 1; “Class” refers to the class of inhibitor described in FIG. 1 and the text. Columns A-D show percent inhibition in the four assays described in FIG. 1. (A) Interphase extracts (B) Mitotic extracts (C) Interphase extracts stimulated with Cdh1 (D) Degradation of a β-catenin reporter protein in interphase extracts stimulated with recombinant axin. Note that in some cases values for percent inhibition are negative; this reflects stimulation of proteolysis by the compound. “ID#” refers to the identification number of the inhibitor obtained from the NCI (prefaced by NSC) or Chembridge (prefaced by C). CAS refers to the chemical abstract services number and is included where available; NA indicates that a CAS number was not available.

The structures of the compounds of the present invention lend themselves readily to efficient synthesis. The nature of the structures of the subject compounds, as generally set forth above, allows the rapid combinatorial assembly of such compounds. For example, solid phase routes can be employed to rapidly assemble a wide variety of structures of Formulae I for testing in the assays described herein. The structures of certain subject ubistatins are well suited for such an approach, because the moieties A, B, L¹, and L², or subsets thereof, can be readily attached using reactions such as those disclosed in de Meijere, A.; Diederich, F. Meta-Catalyzed Cross-Coupling Reactions, 2^{nd} ed.; Wiley and Sons: New York, 2004; Larock, R. C. Comprehensive Organic Transformations, 2^{nd} ed.; Wiley-VCH: New York, 1999; Jung, G. Combinatorial Chemistry: Synthesis, Analysis, Screening, Wiley-VCH: Weinheim, 1999; and Bannwarth, W.; Felder, E. Combinatorial Chemistry: A Practical Approach (Methods and Principles in Medicinal Chemistry), 1^{st} ed.; Wiley-VCH: Weinheim, 2000.

These reactions generally are quite mild and have been successfully applied in combinatorial solid-phase synthesis schemes. Furthermore, the wide range of substrates and coupling partners suitable and available for these reactions permits the rapid assembly of large, diverse libraries of compounds for testing in assays as set forth herein. For certain schemes, and for certain substitutions on the various substituents of the subject compounds, one of skill in the art will recognize the need for masking certain functional groups with a suitable protecting group. Such techniques are well known in the art and are easily applied to combinatorial synthesis schemes.

In addition to the coupling steps described in the above references, additional steps may be used to elaborate or functionalize the basic structural units A, B, L¹ and L², while the structures are bound to the solid support. Furthermore, many variations on the above and related pathways permit the synthesis of widely diverse libraries of compounds that can be tested as therapeutics similar or analogous to those disclosed herein. Simply for illustration, a combinatorial library for the purposes of the present invention is a mixture or set of chemically related compounds that may be screened together for a desired property. The preparation of many related compounds in a single reaction greatly reduces and simplifies the number of screening processes that need to be carried out. Screening for the appropriate physical properties can be done by conventional methods.

Diversity in the library can be created at a variety of different levels. For instance, aryl groups that may be used in the combinatorial reactions can be diverse in terms of the core aryl moiety, e.g., a variegation in terms of the ring structure, and/or can be varied with respect to the other substituents.

A variety of techniques are available in the art for generating combinatorial libraries of small organic molecules such as the subject compounds. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; the ArQule U.S. Pat. Nos. 5,736,412 and 5,712,171; Chen et al. (1994) JACS 116:2661: Kerr et al. (1993) JACS 115:252; PCT publications WO92/10092, WO93/09668 and WO91/07087; and the Lerner et al. PCT publication WO93/20242). Accordingly, a variety of libraries on the order of about 100 to 1,000,000 or more compounds of a general formula, such as that depicted in Formula I, can be synthesized and screened for particular activity or property, such as those described herein.

In one example, a library of candidate compounds can be synthesized utilizing a scheme adapted to the techniques described in the Still et al. PCT publication WO 94/08051, e.g., being linked to a polymer bead by a hydrolyzable or photolyzable group, optionally located at one of the positions of the candidate compounds or a substituent of a synthetic intermediate. According to the Still et al. technique, the library is synthesized on a set of beads, each bead including a set of tags identifying the particular compound on that bead.

The term “acylamino” is art-recognized and refers to a moiety that can be represented by the general formula: 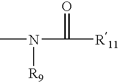
wherein R₉ is as defined above, and R′₁₁ represents a hydrogen, an alkyl, an alkenyl or —(CH₂)ₙ—R₈, and R₈ represents an aryl, a cycloalkyl, a cycloalkenyl, a heterocycle or a polycycle; and n is zero or an integer in the range of 0 to 3.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₙ—R₈, where m and R₈ are described above.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₙ—R₈, wherein n and R₈ are defined above. Representative alkylthio groups include methylthio, ethylthio, and the like.

The terms “amine” and “amino” are art-recognized and refer to both unsubstituted and substituted amines, e.g., a moiety that can be represented by the general formula: 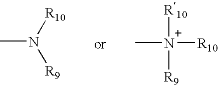
wherein R₉, R₁₀ and R′₁₀ each independently represent a hydrogen, an alkyl, an alkenyl, or —(CH₂)ₙ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and n is an integer in the range of 0 to 3.

The term “amido” is art-recognized as an amino-substituted carbonyl and includes a moiety that can be represented by the general formula: 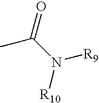
wherein R₉, R₁₀ are as defined above. Preferred embodiments of the amide will not include imides which may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene (phenyl), pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls, for example naphthyl groups.

The term “carbocycle”, as used herein, refers to an aromatic or non-aromatic ring in which each atom of the ring is carbon.

The term “carbonyl” is art-recognized and includes such moieties as can be represented by the general formula: 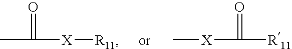
wherein X is a bond or represents an oxygen or a sulfur, and R₁₁ represents a hydrogen, an alkyl, an alkenyl, —(CH₂)ₙ—R₈ or a pharmaceutically acceptable salt, R′₁₁ represents a hydrogen, an alkyl, an alkenyl or —(CH₂)ₙ—R₈, where n and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁′ is hydrogen, the formula represents a “thiocarboxylic acid.” Where X is a sulfur and R₁₁′ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles, for example benzimidazole, benzofuran, and naphthotriazole. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

A “phosphonamidite” can be represented in general formula: 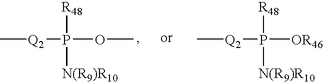
wherein R₉ and R₁₀ are as defined above, Q₂ represents O, S or N, and R₄₈ represents a lower alkyl or an aryl, Q₂ represents O, S or N.

A “phosphoramidite” can be represented in general formula: 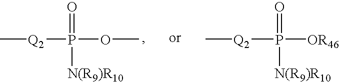
wherein R₉ and R₁₀ are as defined above, and Q₂ represents O, S or N.

A “phosphoryl” can in general be represented by the formula: 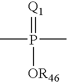
wherein Q₁ represented S or O, and R₄₆ represents hydrogen, a lower alkyl or an aryl. When used to substitute, for example, an alkyl, the phosphoryl group of the phosphorylalkyl can be represented by the general formula: 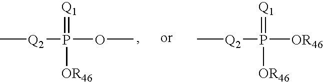
wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “prodrug” is intended to encompass compounds which, under physiological conditions, are converted into the therapeutically active agents of the present invention. A common method for making a prodrug is to include selected moieties which are hydrolyzed under physiological conditions to reveal the desired molecule. In other embodiments, the prodrug is converted by an enzymatic activity of the host animal.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₙ—R₈, n and R₈ being defined above.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula: 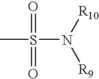
in which R₉ and R₁₀ are as defined above.

The term “sulfate” is art recognized and includes a moiety that can be represented by the general formula: 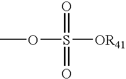
in which R₄₁ is as defined above.

The term “sulfonamido” is art recognized and includes a moiety that can be represented by the general formula: 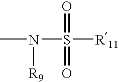
in which R₉ and R′₁₁ are as defined above.

The term “sulfonate” is art-recognized and includes a moiety that can be represented by the general formula: 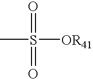
in which R₄₁ is an electron pair, hydrogen, alkyl, cycloalkyl, or aryl.

The terms “sulfoxido” or “sulfinyl”, as used herein, refers to a moiety that can be represented by the general formula: 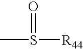
in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

In certain aspects, compounds disclosed herein, and preferably ubistatins, may be used in a combination therapy with, for example, another drug known to have anticancer effects or another drug that inhibits the proteasome pathway. As demonstrated herein, ubistatins have a synergistic effect on the proteasome-mediated protein degradation pathway. Accordingly, a preferred combination therapy comprises a ubistatin and an inhibitor of proteasome-mediated degradation, preferably an inhibitor that acts directly on the proteasome, such as bortezomib (VELCADE), epoxomicin, and YU-101 (Elofsson, M., Splittgerber, U., Myung, J., Mohan, R. & Crews, C. M. (1999) Chem. Biol. 6, 811-822). An alternative combination comprises any of the cell cycle inhibitors disclosed herein and a ubistatin or an inhibitor that acts directly on the proteasome.

Combination therapies may be administered in any manner that takes advantage of the benefits of combination. Typically, the combined agents will be pre-mixed as a single formulation, however, the combined agents may also be administered separately. Where administered separately, the agents may be administered simultaneously or at different times, so long as some benefit of combination is retained.

In further embodiments, a combination therapy may include a ubistatin or other cell cycle inhibitor described herein and another known anticancer agent. A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

Chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

Certain subject compounds may contain a basic functional group, such as amino or alkylamino, and are, thus, capable of forming pharmaceutically acceptable salts with pharmaceutically acceptable acids. The term ‘pharmaceutically acceptable salts’ in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term ‘pharmaceutically acceptable salts’ in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water. In certain embodiments, the pharmaceutical preparation is non-pyrogenic, i.e., does not elevate the body temperature of a patient.

The phrase “therapeutically effective amount” as used herein means that amount of a compound, material, or composition comprising a compound of the present invention which is effective for producing some desired therapeutic effect in at least a sub-population of cells in an animal and thereby blocking the biological consequences of that pathway in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The various compositions described herein may be used to inhibit cell proliferation in vivo or in vitro. Such compositions will be particularly beneficial in the treatment of disorders characterized by abnormal or undesirable cellular proliferation, termed “hyperproliferative” disorders. Examples of hyperproliferative disorders include cancers such as multiple myeloma, a variety of hematologic cancers, solid tumors, pancreatic cancer, prostate cancer, colon cancer, esophageal cancer, breast carcinoma, breast adeno-carcinoma, thyroid carcinoma, liver cancer (hepatocellular cancer), lung cancer, cervical cancer, ovarian cancer, renal cell carcinoma, lymphoma, osteosacoma, liposarcoma, leukemia. The ubistatins and cell cycle inhibitors disclosed herein may be used in the manufacture of a medicament for the treatment of cancers and other hyperproliferative disorders.

The application will be more readily understood by reference to the following examples, which are included merely for purposes of illustration of certain aspects and embodiments of the present application, and are not intended to limit the application.

### EXAMPLES

To identify new components, mechanisms or drug targets in the core cell cycle machinery, applicants developed a high-throughput screen to monitor activation of the cyclin B ubiquitin ligase, the Anaphase-Promoting Complex/Cyclosome (APC/C), in Xenopus cell cycle extracts (3). Because APC/C activation requires mitotic entry, and APC/C activity is required for mitotic exit, monitoring the destruction of APC/C substrates provides a simple means to detect inhibition of cell cycle progression. Moreover, because mitotic entry and exit are not subject to checkpoint regulation in the absence of added nuclei in Xenopus extracts (4, 5), the strategy should yield compounds that inhibit core components of the cell cycle oscillator.

To generate a reporter protein that was sensitive to APC/C activity, the N-terminal 97 amino acids of Xenopus cyclin B1 was fused to luciferase (cyc-luc)(see description of materials and methods below). Quantitative luminometric assays confirmed that cyc-luc was stable in interphase extracts but rapidly degraded in mitotic extracts. Loss of cyc-luc activity in mitosis was impeded by methyl ubiquitin and partially inhibited by the proteasome inhibitor MG132, but not affected by inhibitors of DNA replication or spindle assembly. Activation of latent APC/C activity in interphase extract was blocked by the Cdk1 inhibitor roscovitine; however, roscovitine was ineffective in mitotic extracts that contained activated APC/C. These results confirmed that degradation of the cyc-luc reporter required Cdk1-dependent entry into mitosis, and was mediated by the ubiquitin-proteasome system (UPS).

109,113 compounds were screened for the ability to inhibit cyc-luc turnover using a miniaturized assay system (6). Active compounds were then rescreened in four assays to characterize their mechanism of action. The compounds were first retested in the original assay in which compounds and reporter protein were added to interphase extracts, prior to mitotic entry and APC/C activation. FIG. 1, column A, shows the relative activities of the twenty-two most active compounds whose structures were also confirmed. Next, to distinguish compounds that blocked entry into mitosis from those that had a more direct effect on proteolysis, applicants employed extracts that were stably arrested in mitosis prior to addition of the compound and reporter protein. Sixteen compounds (Class I; see structures above) were no longer inhibitory in this assay, whereas six compounds (Class II; see structures above) retained inhibitory activity (FIG. 1, column B). These findings indicate that Class I compounds inhibited cyc-luc proteolysis by blocking mitotic entry, whereas Class II compounds permitted mitotic entry but blocked cyc-luc proteolysis at a step within mitosis. Consistent with this hypothesis, Class I compounds prevented mitotic phosphorylation of the APC/C subunit Cdc27, whereas Class II compounds did not. To further define the mechanism of inhibition, applicants tested the ability of compounds to inhibit cyclin proteolysis in interphase extracts supplemented with recombinant Cdh1 (FIG. 1, column C). Cdh1 can directly activate the APC/C in interphase extracts without a requirement for mitotic APC/C phosphorylation (7). Interestingly, all of the Class II compounds, but none of the Class I compounds, inhibited Cdh1-stimulated cyc-luc proteolysis. Collectively, these data indicate that Class I compounds blocked entry into mitosis and/or activation of APC/C, whereas Class II compounds either blocked the function of activated APC/C, or acted on a downstream target such as the proteasome.

To simultaneously assess the specificity of inhibition of all compounds and distinguish between the different possible modes of action for Class II compounds, applicants examined turnover of a β-catenin reporter protein (8), a substrate of the SCFβ-TRCP ubiquitin ligase (FIG. 1, column D). Four of the Class I compounds (Class IB) and three of the Class II compounds (Class IIB) inhibited turnover of a catenin-luciferase reporter, suggesting these compounds either inhibit multiple proteins or inhibit a single protein required for the degradation of both APC/C and SCFβ-TRCP substrates. For the Class IB compounds, the most straightforward conclusion is that they inhibited a component that is shared by distinct degradation pathways in the UPS. However, Class IIB compounds did not block either cyclin B ubiquitination in a reconstituted system or 20S peptidase activity in crude extracts, suggesting they did not block the activity of E1 or E2 enzymes or act as conventional proteasome inhibitors.

To understand how the enigmatic Class IIB compounds inhibited proteolysis, applicants turned to a reconstituted system utilizing purified 26S proteasomes and ubiquitinated Sic1 (UbSic1) as the substrate (9). Sic1 is a Cdk inhibitor in Saccharomyces cerevisiae that must be degraded for cells to enter S-phase (9-11). Degradation of Sic1 requires its ubiquitination by the ligase SCFCdc4 (12, 13), following which UbSic1 is docked to the 19S regulatory particle by a multiubiquitin chain receptor (14). Proteolysis of UbSic1 requires removal of the multi-Ub chain, catalyzed by the metalloisopeptidase Rpn11 (15, 16). The deubiquitinated substrate is then translocated into the 20S core particle where it is degraded.

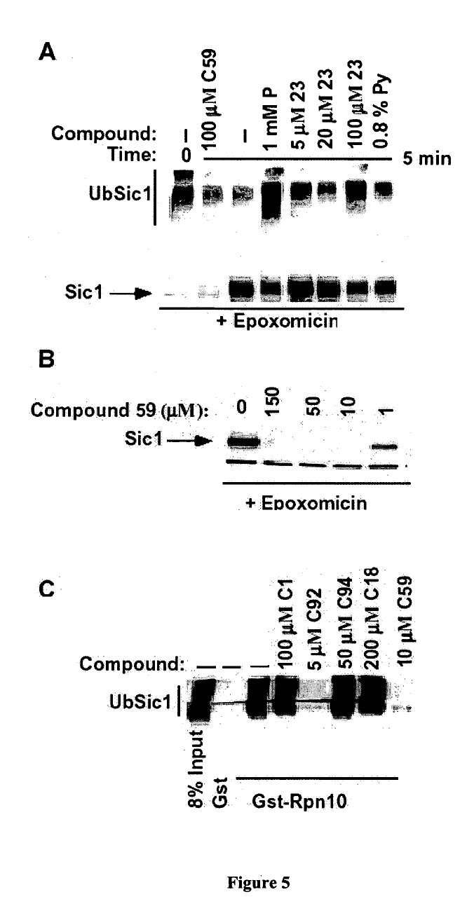

Analysis of UbSic1 turnover in this purified system revealed that two Class IIB molecules, compound 92 (C92) and compound 59 (C59), had a strong inhibitory effect on Sic1 turnover even at concentrations as low as 10 μM, whereas compound 23 had a more modest effect (FIG. 2A). To address whether these compounds acted upstream or downstream of Rpn11 isopeptidase, applicants evaluated deubiquitination of UbSic1. Deubiquitination can be revealed in vitro when proteolysis is blocked with the 20S core protease inhibitor epoxomicin (15, 17), resulting in accumulation of deubiquitinated Sic1 within the 20S chamber (14). Incubation of UbSic1 with epoxomicin-treated proteasomes led to deubiquitination of the substrate, but this reaction was completely blocked by C92 (FIG. 2B). Titration of C92 in this assay revealed an IC50 of approximately 400 nM (FIG. 2C). Of the two remaining Class IIB compounds C59, which is structurally related to C92, also inhibited deubiquitination of UbSic1 (IC50 1 μM), whereas C23 inhibited marginally (FIGS. 5A and B). From this analysis we conclude that C92 and C59 potently block proteolysis at, or upstream of, the essential isopeptidase-dependent step.

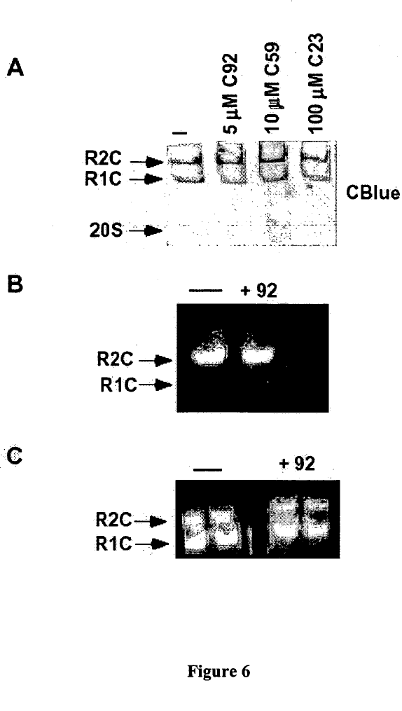

Selective recognition of the multiubiquitin chain by the 26S proteasome constitutes the first step in UbSic1 degradation (9). Applicants therefore assayed the binding of UbSic1 to 26S proteasomes pre-treated with C92. The results in FIG. 2D demonstrate that C92 strongly inhibited binding of UbSic1 to purified 26S proteasomes. Previous work by applicants has shown that multiubiquitin chain receptors Rad23 and Rpn10 serve a redundant role in sustaining UbSic1 degradation in vitro and in vivo (14). In the absence of the ubiquitin-binding activity of Rpn10, UbSic1 is not recruited, deubiquitinated, or degraded by purified 26S proteasomes. Thus, to further establish the mechanism by which C92 inhibited binding of UbSic1 conjugates to the proteasome, applicants tested whether C92 could interfere with binding of UbSic1 to recombinant Rpn10 and Rad23. Remarkably, C92 abolished binding of UbSic1 to both proteins (FIG. 2E), even though these receptors employ distinct domains (the ubiquitin-interaction motif [UIM] and the UBA domain, respectively) to bind ubiquitin chains (18). Additional analysis revealed that C59, like C92, also abrogated binding of UbSic1 to Rpn10, whereas other compounds were without effect (FIG. 5). To distinguish whether C92 inhibited proteolysis by binding to proteasome receptor proteins or to the Ub chain on Sic 1, applicants exploited the negative charge of C92 to determine whether compound binding induced a mobility shift of the target proteins on native gels. C92 was pre-incubated with recombinant Rpn10, Rad23, or a mixture of Ub chains containing 2-7 Ub molecules. The data in FIG. 2F demonstrate that the mobility of the multiUb chains, but not Gst-Rpn10 or Gst-Rad23, was altered by incubation with C92, indicating that C92 bound Ub chains. Remarkably, the binding of C92 and C59 to ubiquitin chains was exquisitely linkage specific. Ubiquitin molecules can be linked to each other in vivo through different internal lysines, including K29, K48, and K63(19). The K48-linked chain is the principal targeting signal in proteolysis whereas K63-linked chains are implicated in enzyme regulation; no physiological function for K29-linked chains has yet been discovered (20). Whereas C92 and C59 efficiently shifted the native gel mobility of K48-linked ubiquitin chains, they had little or no effect on K29- or K63-linked chains (FIG. 2G). Because C92 and C59 bind to ubiquitin chains and block interactions with proteasome-associated receptors without affecting 26S assembly or peptidase activity (FIG. 6), applicants termed these compounds ubistatin A and B, respectively.

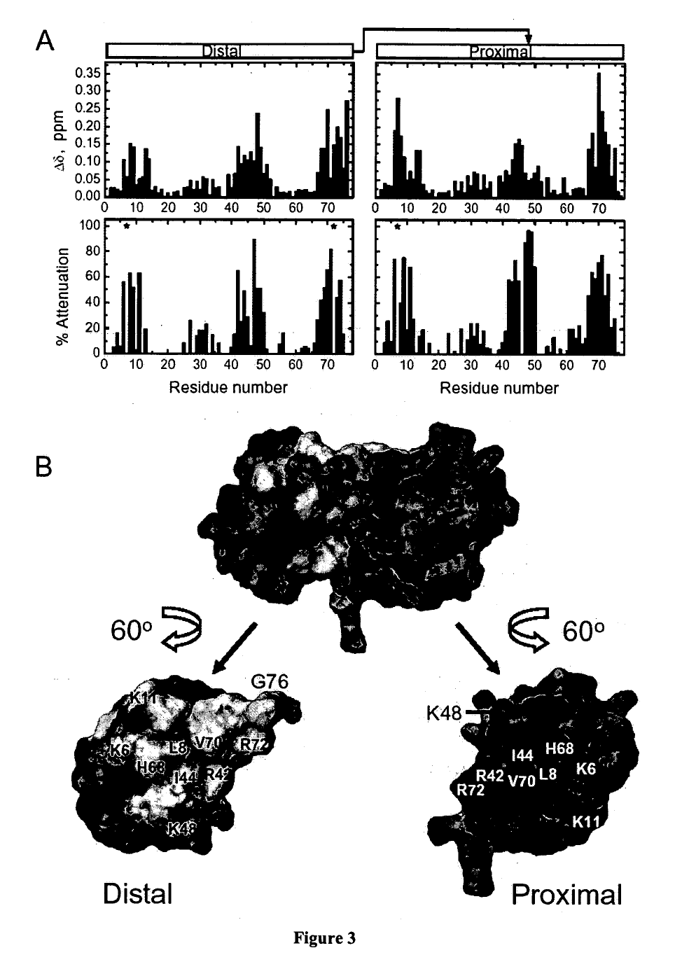

The high specificity of ubistatin A for K48-linked ubiquitin chains suggested that it might bind at the Ub-Ub interface, which is well-defined in K48-linked chains but is not present in K63-linked di-ubiquitin (21). To determine if ubistatin A influenced this unique interface, NMR titration studies of K48-linked di-ubiquitin (Ub2) were performed using a segmental labeling strategy in which only one Ub unit per chain was isotopically enriched (22). This revealed perturbations or interactions specific for each Ub domain in Ub2. NMR titration data indicated well-defined site-specific perturbations in the resonances of the backbone amides of both Ub units in Ub2 (FIG. 3). These perturbations increased upon addition of ubistatin A and saturated at the ubistatin:Ub2 molar ratio of ˜3:1. Based on the observed chemical shift perturbations and signal attenuations it was apparent that the “hydrophobic patch” residues L8, I44, V70, and neighboring sites (including basic residues K6, K11, R42, H68, R72 that may interact with sulfonates of ubistatin A) experienced alterations in their molecular environment upon binding of ubistatin A. Interestingly, the same hydrophobic patch is involved in the formation of the interdomain interface in Ub2 (23, 24), and mediates the binding of ubiquitin to multiple proteins containing CUE, UBA, and UIM domains (18). This striking observation suggests that ubistatin A either induces a conformational rearrangement in Ub2 that changes the functional properties of the ubiquitin chain, or directly competes out the binding of ubiquitin receptors through a simple steric effect. At the high concentrations of compound used in the NMR titration experiments, ubistatin A induced a similar pattern of chemical shift perturbations in monomeric ubiquitin, suggesting that the effect of ubistatin A on di-ubiquitin arises from its direct binding to the hydrophobic patch and the basic residues around it. The same sites are perturbed when ubistatin A binds tetra-ubiquitin chains. Interestingly, the structure of ubistatins, consisting of a series of negative charges on a relatively hydrophobic core, mimics the consensus sequence of the UIM motif that recognizes K48-linked ubiquitin chains (Φ-x-x-Ala-x-x-x-Ser-x-x-Ac, in which Φ denotes a large hydrophobic residue and ‘Ac’ denotes an acidic residue) (24).

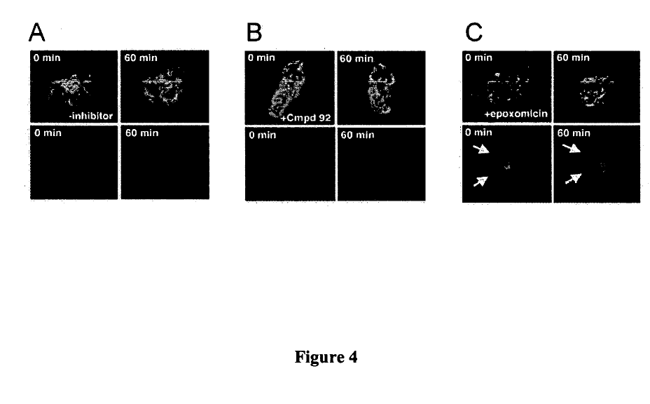

The effect of ubistatin A on protein degradation within intact mammalian cells was also evaluated (FIG. 4). Since the negative charge of the molecule was predicted to preclude efficient entry into the cell, compound was introduced by microinjection. Previous results established that Protacs, comprised of a ubiquitin ligase-binding peptide from IκB linked to a target-binding small molecule, induce degradation of the target in a proteasome-dependent manner by recruiting the target to the SCF ubiquitin ligase (25, 26). Protac-X, which contains dihyroxytestosterone as the target-binding small molecule, induces rapid degradation of GFP-androgen receptor (GFP-AR) upon introduction into cells. HEK293 cells expressing GFP-AR were injected with Protac-X alone or Protac-X plus ubistatin A or epoxomicin, and monitored for presence or absence of GFP by fluorescence microscopy. Protac-X induced rapid loss of GFP-AR without compromising cell integrity (as monitored by retention of rhodamine dye), but this effect was completely blocked by simultaneous introduction of either 100 nM ubistatin A (FIG. 4B) or 100 nM epoxomicin (FIG. 4C). Together the data demonstrate that ubistatin A is an effective inhibitor of ubiquitin-dependent degradation in diverse experimental systems, which is likely a reflection of the highly conserved sequence of the lysine-48 linked ubiquitin chain.

In the current era of rational drug design, there is intense interest in developing drugs whose molecular targets are known such that unwanted side effects can be avoided during therapy. However, it is often difficult to know, a priori, which proteins can be most effectively targeted with small molecules. Unbiased chemical genetic screens provide an unparalleled opportunity to discover new drugable targets within pathways of interest, yet identification of the protein target can be challenging. The present study demonstrates that chemical genetic screens in complex biochemical systems such as Xenopus cell cycle extracts can identify novel small molecule inhibitors that act through unexpected mechanisms. The ability to biochemically manipulate these extracts allowed the subdivision of the compounds into four different functional classes. To circumvent the difficult problem of target identification, one class of compounds was subjected to a series of secondary assays of decreasing biochemical complexity, leading to identification of the receptor-binding interface of K48-linked ubiquitin chains as the target of one class of inhibitors. The same approach is now being employed to unravel the molecular targets of selected compounds from the other classes unearthed in our primary screen.

A small molecule inhibitor of the 20S proteasome, Velcade, has recently been approved for treatment of relapsed multiple myeloma, and there are multiple ongoing studies to evaluate the efficacy of proteasome inhibitors in other tumors (27). The present unbiased screen identified ubistatins as an entirely unexpected class of inhibitors of proteasome-dependent degradation that operate by inhibiting a protein-protein interaction. The identification of the ubiquitin chain as a drugable target in the ubiquitin system suggests rational strategies for the identification of second-generation ubistatins with improved drug-like properties.

Preparation of Proteins for Screening

To construct a cyclin-luciferase fusion protein (pSP cyc-luc), the N-terminal sequence of Xenopus laevis cyclin B1, including amino acids 2-97, was amplified by PCR, digested with BstEII, and ligated into the pSP-lucNF expression vector (Promega). The resulting vector was sequence verified. The fusion protein was expressed by coupled in vitro transcription and translation in reticulocyte lysate using the SP6-TNT Coupled Reticulocyte Lysate System (Promega) and flash frozen in liquid nitrogen until the time of use. The parental pSP-lucNF vector was used to express unmodified luciferase. A vector for expression of cyclin-luciferase in E. coli (pET cyc-luc) was also constructed; this protein behaved identically in all assays to the protein expressed in reticulocyte lysate, but could be made in higher quantities necessary for screening. pSP cyc-luc was digested with HindIII and XhoI. The resulting 1949 bp fragment containing the cyclin B1-luciferase sequence was ligated into the pET 28b expression vector (Novagen) containing an N-terminal hexahistidine tag for protein purification. To express this fusion protein, one liter of LB containing E. Coli strain BL21(DE3) was grown at 37° C. to an OD600 of 0.6. Expression was induced for 3 hrs with 1 mM IPTG. The cells were pelleted and lysed, and protein purified by Ni-NTA batch purification under native protein conditions (Qiagen). Sea urchin cyclin B90 was prepared as described (29). Methylated ubiquitin was prepared by reductive methylation of bovine ubiquitin as described (30).

Preparation of Xenopus Egg Extracts

Xenopus egg extracts were prepared from eggs laid overnight according to the protocol of Murray (31) with the exception that eggs were activated with 0.2 μg/ml calcium ionophore (A23187, free acid form, Calbiochem) for forty minutes prior to the crushing spin. Extracts were frozen in liquid nitrogen and stored at −80° C. Eggs laid from 40 frogs typically yielded a total 70 ml of cytoplasmic extract.

Assay Validation

Extracts were rapidly thawed and diluted to a final concentration of 75% in extract buffer (XB) just prior to assay (XB; 100 mM KCl, 0.1 mM CaCl₂, 1 mM MgCl2, 10 mM potassium HEPES, pH 7.7, 50 mM sucrose). Extracts were kept on ice and supplemented with 1 mg/ml bovine ubiquitin (Sigma) unless otherwise stated. Cyc-luc expressed in reticulocyte lysate was diluted 1/200 for most assays. High-throughput screening utilized the bacterially expressed and purified cyclin-luc fusion protein, which was added to extracts at a final concentration of 0.1 μg/ml. To induce entrance into mitosis, cyclin BΔ90 protein was added to a final concentration of 10 μg/ml. For the dose response analysis, inhibitors were purchased from Calbiochem (San Diego, Calif.) and dissolved in DMSO as 10 mM stocks. Cyclin-luciferase and cyclin BΔ90 were mixed with interphase extract and placed on ice. Inhibitors were then added to extracts to yield a final DMSO concentration of 1%. 10 μl aliquots of extract were distributed into 384-well white Cliniplates (Labsystems), and cell cycle progression was initiated by warming the samples to room temperature. After 60 minutes, 30 μl of luciferin reagent (20 mM tricine pH 7.8, 470 μM D-Luciferin [Molecular Probes]), 270 μM Coenzyme A, 0.1 mM EDTA, 33 mM DTT, and 530 μM ATP) was added using a multidrop dispenser (Labsystems). Luminescence was measured on an Analyst plate reader (LJL Biosystems). The values for 3 replicates were averaged. For the dose response analysis, percent inhibition was calculated as 100*(T−M)/(I−M) where T equals the test value for the inhibitor, M equals the value in a mitotic extract lacking inhibitor, and I equals the value in an interphase extract. For certain experiments, methylated ubiquitin was added to interphase extracts at a concentration of 1 mg/ml in the presence or absence of added unmodified ubiquitin (1 mg/ml) used as competitor. Cyclin-luciferase and cyclin BΔ90 were then added. After 60 minutes, the reactions were stopped and analyzed as described above.

Chemical Libraries and Chemical Characterization of Active Compounds

Compound collections screened included 16,320 compounds from Chembridge Corporation (San Diego, Calif.; Diverset E); 1991 compounds from the NCI Diversity set, and 90,802 compounds from the NCI open collection. Compounds that retained activity after retesting were reobtained as dry powders and characterized by LC-MS; only compounds that were greater than 90% pure with an appropriate mass were evaluated in further experiments and presented in FIG. 1.

For ubistatin A (C92), we performed additional structural characterization of a sample of dry powder provided by the NCI (NSC665534). NMR spectra were recorded at ambient temperature in D₂O with a 5 mm probe operating at 500 MHz (₁H) or 75 MHz (₁₃C). For 1H NMR the internal reference was TSP (δ 0.00). 1H NMR (D₂O) δ8.51 (d, J=3.0 Hz, 2H), 8.21 (d, J=2.1 Hz, 2H), 7.98(br, J=8.1 Hz, 2H), 7.80 (m, 6H), 7.60 (dd, J=8.4; 2.1 Hz, 2H) 7.35 (dd, J=8.4; 4.2 Hz, 2H), 6.82 (d, J=8.7 Hz, 2H, H-d). These measurements are identical to those reported in the literature (32). Elemental analysis was also performed (M-H-W laboratories, Phoenix Ariz.): Anal. Calcd. For C₃₄H₂₀N₄Na₄O₁₆S₄; C, 42.50; H, 2.10; N, 5.83. Found: C, 42.65; H, 2.24; N, 5.88.

High-Throughput Screening

Chilled interphase Xenopus extracts containing cyclin B 90 and cyclin-luciferase were spread onto chilled customed-designed 1536-well plates (6) that held 2 microliters of extract per well. 100 nl of compound (5-10 mM in DMSO) was then transferred into the assay plate using a custom-designed pin-transfer robot (6). After a 60 minute incubation at room temperature, a PixSys 3200 dispenser (Cartesian Technologies, Irvine, Calif.) was used to dispense 200 nl of a 10× concentrated luciferin solution to each well of the assay plate. Immediately after filling, the plate was imaged using the Leadseeker system (Amersham Pharmacia Biotech, Amersham, U.K.). Exposure times were typically 2 minutes. Image analysis and quantitation was performed using the MCID Assayvision software (Imaging Research Incorporated, St. Catherines, Canada).

A total of 109,113 compounds were screened in duplicate. A low threshold was set for identification of initial hits, and included compounds that increased the luminescence reading by two-fold above the median value calculated for all wells on the plate. We included compounds that scored on either of the duplicate set of plates, and noted that about 65% of the compounds scored on both plates, whereas 35% were active only on a single plate. This rate of overlap may be a result of variability in the amount of compound transferred. The initial screen identified a total of 1017 hits (247 from the Chembridge Library and the NCI diversity set; 770 from the NCI open collection). In the next step, DMSO stocks of active compounds were selected to create a new master plate. All of the hits from the Chembridge Library and the NCI diversity set were replated, whereas only 444 compounds from the NCI open collection were replated, as a high fraction of compounds were excluded due to chemical or structural considerations (compounds with very simple structures, reactive structures, or which contained metal complexes were excluded). The resulting 691 compounds were then retested in 384-well plates at 200 μM concentration under the conditions described for validation of the assay. In this case, compounds were prediluted in XB prior to addition to extracts by pipetting, assuring more accurate compound concentration. A large number of compounds failed to retest as positive at this point, presumably due to the higher concentrations used in the primary screen.

The 96 most active compounds were then reobtained as dry powders from either Chembridge or the NCI. These compounds were retested in the four assays described in FIG. 1, at 200 μM concentration, as described below. The data in FIG. 1 represent the 22 most active compounds (compounds that showed at least 30% inhibition) whose structures could also be confirmed by LC-MS analysis. Table 1 provides the relevant Chembridge ID or NCI identification numbers for each of these compounds.

Retesting of Active Compounds

Dry compounds were redissolved at 20 mM concentration in DMSO, or a 50:50 water:DMSO mixture in certain cases. These compounds were then diluted ten-fold in XB and mixed thoroughly. For the assay in FIG. 1A, chilled interphase extract was mixed with cyclin BΔ90 (10 μg/ml) and cyc-luc (0.1 μg/ml) in bulk and then 27 μl of extract was pipetted to each well of a chilled 384-well plate. Three microliters of each compound (diluted in XB) was then added to each well, and then compounds mixed thoroughly. Plates were warmed to room temperature. After 70 minutes, 5 μl aliquots were transferred to another plate, and then 30 μl of luciferin reagent was added to each well and luminescence measured. Percent inhibition was calculated as described above. The reported values represent the average of three independent measurments. For the assay in FIG. 1B, interphase extracts were treated with 10 μg/cyclin B 90 for 50 minutes at room temperature and then chilled on ice. Cyc-luc was then added, and then 27 μl of extract was pipetted to each well of a chilled 384-well plate. Three microliters of each compound (diluted in XB) was then added to each well, and then compounds mixed thoroughly. Plates were warmed to room temperature. After 60 minutes, 5 μl aliquots were transferred to another plate, and then 30 μl of luciferin reagent was added to each well and luminescence measured. For the assay in FIG. 1C, recombinant Cdh1 (0.1 μg/ml) and cyc-luc were added to chilled interphase extracts. Extracts were then pipetted and compounds added as above. Plates were warmed to room temperature and incubated for three hours, following which luminescence was measured as above. For the assay in FIG. 1D, chilled interphase extracts received recombinant axin and beta-catenin-luciferase reporter as described (8). Extracts were aliquoted to chilled plates and compounds distributed as described above. Plates were warmed to room temperature, incubated for three hours, and luminescence measured.

Preparation of UbSic1

Sic1, expressed in E. coli as a Maltose-binding-protein chimera tagged at the C-terminus with the MycHis6 tag (MbpSic1_{mycHis6}) was purified as described (10). It was phosphorylated and ubiquitinated utilizing insect expressed kinase and SCFCdc4 complexes as described (13). Ubiquitinated MbpSic1_{mycHis6} is designated UbSic1 throughout this disclosure.

Purification of 26S Proteasomes

26S proteasomes were purified from S. cerevisiae cells expressing a Flag-tagged proteasomal subunit (PRE1) essentially as described (28). Briefly, lysates were immunoaffinity purified on anti-Flag resin in the presence of 2 mM ATP and 5 mM MgCl2, and eluted with Flag peptide.

Degradation of UbSic1

Ubiquitinated Sic1 (˜300 nM) was incubated with purified 26S proteasomes (˜100 nM) at 30° C. for 5 min (28). The reaction tubes were transferred to ice and quenched with 5× Laemmli SDS sample buffer. Aliquots were resolved by SDS-PAGE, transferred to nitrocellulose, and immunoblotted with polyclonal anti-Sic1 Ab.

Deubiquitination of UbSic1

Purified 26S proteasomes were preincubated with 100 uM epoxomicin at 30° C. for 45 min before addition of UbSic1 (15). Reactions were processed as above.

Purification of Gst-Fusion Proteins

Recombinant proteins were purified as described (14).

Binding of UbSic1 to 26S Proteasomes

26S proteasomes were immunoprecipitated with anti-Flag resin from tagged and untagged control strains and incubated with 1 mM phenanthroline, 2.5 uM Ub-aldehyde, and 100 μM MG132 at 4° C. for 45 min. UbSic1 was then added in binding buffer containing 25 mM Tris, pH 7.5, 1 mM ATP, 100 mM NaCl, 5 mM MgCl2, 0.2% Triton in the presence or absence of 5 μM C92. Following binding, beads were washed twice with same buffer, and once with 25 mM Tris, 5 mM MgCl2, 1 mM ATP. Washed beads were resuspended in 2×SDS sample buffer and aliquots analyzed by immunoblotting with anti-Sic1.

Binding of UbSic1 to Gst-Fusion Proteins

Recombinant Gst-Rpn10 and Gst-Rad23 were immobilized on glutathione sepharose beads. UbSic1 was added in binding buffer containing 25 mM Tris, pH 7.5, 150 mM NaCl and 0.2% Triton in the presence or absence of C92. Following binding at 4° C. for 90 mins, the beads were centrifuged, and washed twice with the same buffer, and twice with buffer containing 25 mM Tris, pH 7.5. They were then resuspended in an equal volume of 2×SDS sample buffer. Aliquots were analyzed by immunoblotting.

NMR Studies

Synthesis of segmentally ₁₅N-labeled Ub₂ chains was performed as described in (22). Ubiquitin monomers and C170S-E225K were expressed and purified as described in (33). ₁₅N-labeled Ub (D77) and Ub (K48C) were expressed in E. coli cells grown in minimal media with ₁₅NH₄Cl as the sole source of nitrogen. E1 and Ub C-terminal hydrolase were from BostonBiochem Inc., and ethyleneimine was from Chemservice.

All NMR measurements were performed at 24° C. on a Bruker DRX spectrometer operating at ₁H resonance frequency of 600.13 MHz. The protein samples (concentration range from 0.1-0.6 mM) were prepared in 20 mM phosphate buffer, pH 6.8, containing 7% D₂O and 0.02% NaN₃. ₁H-₁₅HSQC spectra were acquired with spectral widths of 7.2 kHz and 2 kHz in the ₁H and ₁₅N dimensions, respectively. For each 2D plane, 128 t₁ increments were collected, each consisting of 1024 complex points.

For NMR titration studies ubistatin A was added to the di-ubiquitin sample in small steps from a 1 mM stock solution in the same buffer as the protein. The titration continued up to a molar ratio, ubistatin:Ub₂, of 4:1 (distal-domain-labeled Ub₂), 5:1 (proximal-domain-labeled Ub₂). Binding of ubistatin A was monitored by signal attenuation and shifts in the resonance peak positions of the backbone amides in each Ub domain. The combined amide chemical shift perturbations were computed as Δδ=[(Δδ_{H})₂+(Δδ_{N}/5)₂]_{1/2}, where Δδ_{H} and Δδ_{N} are the chemical shift differences (for ₁H and ₁₅N, respectively) between the free and ubistatin A-bound di-ubiquitin for a given amide group. The signal attenuation for each residue was calculated as (1−I/Iₒ)*100%, where Iₒ and I are peak intensities in HSQC spectra of the free and C92-bound protein; the latter values were uniformly scaled to account for higher molecular weight of the complex and for the differences in the protein concentration and the experimental settings between the experiments.

Microinjection Experiments

HEK-293 cells stably transfected with a plasmid that expresses GFP-AR were microinjected with 0.2 pl (5-10% of cell volume) of a 200 mM KCl solution containing 10 μM Protac and 50 mg/ml rhodamine dextran (MW 10,000 Da). For C92 and proteasome inhibition experiments, cells were co-injected with 1 μM C92 or epoxomicin (yielding a final intracellular concentration of 50-100 nM) and Protac (10 μM) as previously described (26).

### REFERENCES

1. T. U. Mayer, Trends Cell Biol. 13, 270-277 (2003).
2. C. Crews, J. Shotwell, Prog. Cell Cycle Res. 5, 125-33 (2003).
3. J. M. Peters, Mol. Cell 9, 931-43 (2002).
4. J. Minshull, H. Sun, N. K. Tonks, A. W. Murray, Cell 79, 475-86 (1994).
5. M. Dasso, J. W. Newport, Cell 61, 811-23 (1990).
6. L. A. Walling, N. R. Peters, E. J. Horn, R. W. King, J. Cell. Biochem. S37, 7-12 (2001).
7. C. M. Pfleger, M. W. Kirschner, Genes Dev. 14, 655-65 (2000).
8. A. Salic, E. Lee, L. Mayer, M. W. Kirschner, Mol. Cell 5, 523-32 (2000).
9. R. Verma, H. McDonald, J. R. Yates, 3rd, R. J. Deshaies, Mol. Cell 8, 439-48 (2001).
10. R. Verma et al., Science 278, 455-60 (1997).
11. E. Schwob, T. Bohm, M. D. Mendenhall, K. Nasmyth, Cell 79, 233-44 (1994).
12. D. Skowyra et al., Science 284, 662-5 (1999).
13. J. H. Seol et al., Genes Dev. 13, 1614-26 (1999).
14. R. Verma, R. Oania, J. Graumann, R. J. Deshaies, Cell, in press.
15. R. Verma et al., Science 298, 611-5 (2002).
16. T. Yao, R. E. Cohen, Nature 419, 403-7 (2002).
17. L. Meng et al., Proc. Natl. Acad. Sci. USA 96, 10403-8 (1999).
18. R. Hartmann-Petersen, M. Seeger, C. Gordon, Trends Biochem. Sci. 28, 26-31 (2003).
19. J. Peng et al., Nat. Biotechnol 21, 921-6 (2003).
20. C. M. Pickart, Cell 116, 181-90 (2004).
21. R. Varadan et al., J Biol Chem 279, 7055-63 (2004).
22. R. Varadan, O. Walker, C. Pickart, D. Fushman, J. Mol. Biol. 324, 637-47 (2002).
23. W. J. Cook, L. C. Jeffrey, M. Carson, Z. Chen, C. M. Pickart, J. Biol. Chem. 267, 16467-71 (1992).
24. K. Hoffman, L. Falquet, Trends Biochem. Sci 26, 347-50 (2001).
25. K. M. Sakamoto et al., Proc. Natl. Acad. Sci. USA 98, 8554-9 (2001).
26. K. M. Sakamoto et al., Mol. Cell. Proteomics 2, 1350-1358 (2003).
27. J. Adams, Nat. Rev. Cancer 4, 349-60 (2004).
28. R. Verma et al., Mol. Biol. Cell 11, 3425-39 (2000).
29. M. Glotzer, A. W. Murray, M. W. Kirschner, Nature 349, 132-8 (1991).
30. A. Hershko, H. Heller, Biochem. Biophys. Res. Commun. 128, 1079-86 (1985).
31. A. W. Murray, Methods Cell. Biol. 36, 581-605 (1991).
32. R. D. Haugwitz, L. Zalkow, E. Gruszecka-Kowalik, E. Burgess, “Aryolaniline compounds, pharmaceutical compositions, and methods of using same to inhibit viral activity” (U.S. Pat. No. 5,681,832, 1997).
33. M. T. Haldeman, G. Xia, E. M. Kasperek, C. M. Pickart, Biochemistry 36, 10526-37 (1997).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method of inhibiting proteosomal degradation of a multiubiquitinated protein in a cell, the method comprising contacting the cell with a ubistatin that binds to a K48-linked multiubiquitin chain and inhibits proteasome-based degradation of a polypeptide that is covalently bound to a K48-linked multiubiquitin chain.

2. The method of claim 1, wherein the ubistatin causes half-maximal inhibition of proteasome-mediated degradation in an in vitro assay at a concentration of about 500 nM or less.

3. The method of claim 2, wherein the ubistatin causes greater than 50% inhibition of the binding of a K48-linked multiubiquitin chain to one or more multiubiquitin binding proteins, such as Rpn10 or Rad23 at concentrations of 5 μM or less.

4. The method of claim 3, wherein the ubistatin binds to a set of amino acid residues on the Ub-Ub interface of the K48-linked chain including at least one amino acid selected from the group consisting of: L8, I44, V70, K6, K11, R42, H68 and R72.

5. The method of claim 3, wherein the compound comprises a purified organic compound having a molecular weight less than 2000 amu, wherein the compound has the Formula (I):
A¹-L¹-B¹-M-B²-L²-A² (I)
or a pharmaceutically acceptable salt or prodrug thereof, wherein as valence and stability permits:
L¹, L², and M independently for each occurrence, represent a direct bond, —(CR¹R²)ₙ—, —(CR¹═CR²)ₙ—, —O—, —S—, —Se—, —NR³—, —(N═N)ₙ—, —O—N═CH—, —(R₃)N—N(R₃)—, —O—N(R₃)—, —NR³—C(O)—, —C(O)—NR³—, —C(O)—, —C(S)—, —O—C(O)—O—, —O—C(S)—O—, —NR³—C(O)—NR³—, —O—C(O)—, —C(O)—O—, —NR³—C(S)—NR³—, —O—C(S)—, —C(S)—O—, —S(O)ₘ—, or —C(O)—C(O)—; and
A¹, A², B¹ and B² independently for each occurrence, are absent or represent aryl, heteroaryl, cycloalkyl, cycloalkenyl, heterocyclyl, aralkyl, heteroaralkyl, or polycyclyl, with 1-3 substitutions of R^{a} and 0-3 substitutions of R^{b};
wherein any one of A¹, A², B¹ and B² are optionally connected to any one of the others by one or more bonds;
wherein R^{a}, independently for each occurrence, represents —S(O)ₘ—OH, —CO₂H, —P(O)—(OH)ₘ, —OH, or a moiety having an ionizable hydrogen and a pKa of less than 10;
R^{b}, independently for each occurrence, represents one or more of hydrogen, halogen, hydroxyl, alkoxyl, silyloxyl, amino, nitro, sulfhydryl, alkylthio, imino, amido, cyano, carbonyl, carboxyl, silyl, sulfamoyl, sulfinyl, thioalkyl, alkylsulfonyl, arylsulfonyl, acyl, formyl, esteryl, isocyano, guanidinyl, amidinyl, acetalyl, ketalyl, amine oxidyl, azido, carbamyl, hydroxamyl, imidyl, oximyl, sulfonamidyl, thioamidyl, thiocarbonyl, ureayl, thioureayl, or a substituted or unsubstituted alkyl or, alkenyl, alkynyl, aryl or heteroaryl or —(CH₂)ₙ—R₄;
R¹, R², R³, independently for each occurrence, represent hydrogen, alkoxy, halogen, lower alkyl (substituted or unsubstituted), aryl (substituted or unsubstituted), aralkyl (substituted or unsubstituted), heteroaryl (substituted or unsubstituted), or heteroaralkyl (substituted or unsubstituted);
R₄, independently for each occurrence, represents a substituted or unsubstituted aryl, cycloalkyl, cycloalkenyl, heterocyclyl, heteroaryl, or polycyclyl;
m, independently for each occurrence, represents an integer from 1 to 2; and
n, independently for each occurrence, represents an integer from 0 to 3.

6. The method of claim 5 wherein M is —(CR¹═CR²)ₙ—, —(N═N)ₙ—, or —(R₃)N—N(R₃)—, and n is 1.

7. The method of claim 5 wherein B¹ and B² is aryl or heteroaryl.

8. The method of claim 5 wherein L¹ and L² is a direct bond.

9. The method of claim 5 wherein L¹ and L² is —NR³—C(O)— or —C(O)—NR³—.

10. The method of claim 5 wherein A¹ and A² is aryl, heteroaryl, or polycyclyl.

11. The method of claim 5 wherein one or more R^{a} is —S(O)ₘ—OH and wherein m is 2.

12. The method of claim 6 wherein M is —(CR¹═CR²)ₙ—, wherein n is 1 and both R¹ and R² are hydrogen.

13. The method of claim 7 wherein B¹ and B² are phenyl.

14. The method of claim 10 wherein A¹ and A² are naphthyl.

15. The method of claim 10 wherein A¹ and A² are naphthotriazole.

16. The method of claim 5 wherein each instance of A¹, A², B¹ and B² is substituted by at least one R^{a}, wherein R^{a} is —S(O)ₙ—OH and wherein m is 2.

17. A method of treating a cancer in a patient in need thereof, the method comprising administering to the patient a ubistatin that binds to a K48-linked multiubiquitin chain and inhibits proteasome-based degradation of a polypeptide that is covalently bound to a K48-linked multiubiquitin chain.

18. The method of claim 17, wherein the ubistatin causes half-maximal inhibition of proteasome-mediated degradation in an in vitro assay at a concentration of about 500 nM or less.

19. The method of claim 18, wherein the ubistatin causes greater than 50% inhibition of the binding of a K48-linked multiubiquitin chain to one or more multiubiquitin binding proteins, such as Rpn10 or Rad23 at concentrations of 5 μM or less.

20. The method of claim 19, wherein the ubistatin binds to a set of amino acid residues on the Ub-Ub interface of the K48-linked chain including at least one amino acid selected from the group consisting of: L8, I44, V70, K6, K11, R42, H68 and R72.

21. The method of claim 19, wherein the compound comprises a purified organic compound having a molecular weight less than 2000 amu, wherein the compound has the Formula (I):
A¹-L¹-B¹M-B²-L²-A² (I)
or a pharmaceutically acceptable salt or prodrug thereof, wherein as valence and stability permits:
L¹, L², and M independently for each occurrence, represent a direct bond, —(CR¹R²)ₙ—, —(CR¹═CR²)ₙ—, —O—, —S—, —Se—, —NR³—, —(N═N)ₙ—, —O—N═CH—, —(R₃)N—N(R₃)—, —O—N(R₃)—, —NR³—C(O)—, —C(O)—NR³—, —C(O)—, —C(S)—, —O—C(O)—O—, —O—C(S)—O—, —NR³—C(O)—NR³—, —O—C(O)—, —C(O)—O—, —NR³—C(S)—NR³—, —O—C(S)—, —C(S)—O—, —S(O)ₘ—, or —C(O)—C(O)—; and
A¹, A², B¹ and B² independently for each occurrence, are absent or represent aryl, heteroaryl, cycloalkyl, cycloalkenyl, heterocyclyl, aralkyl, heteroaralkyl, or polycyclyl, with 1-3 substitutions of R^{a} and 0-3 substitutions of R^{b};
wherein any one of A¹, A², B¹ and B² are optionally connected to any one of the others by one or more bonds;
wherein R^{a}, independently for each occurrence, represents —S(O)ₘ—OH, —CO₂H, —P(O)—(OH)ₘ, —OH, or a moiety having an ionizable hydrogen and a pKa of less than 10;
R^{b}, independently for each occurrence, represents one or more of hydrogen, halogen, hydroxyl, alkoxyl, silyloxyl, amino, nitro, sulfhydryl, alkylthio, imino, amido, cyano, carbonyl, carboxyl, silyl, sulfamoyl, sulfinyl, thioalkyl, alkylsulfonyl, arylsulfonyl, acyl, formyl, esteryl, isocyano, guanidinyl, amidinyl, acetalyl, ketalyl, amine oxidyl, azido, carbamyl, hydroxamyl, imidyl, oximyl, sulfonamidyl, thioamidyl, thiocarbonyl, ureayl, thioureayl, or a substituted or unsubstituted alkyl or, alkenyl, alkynyl, aryl or heteroaryl or —(CH₂)ₙ—R₄;
R¹, R², R³, independently for each occurrence, represent hydrogen, alkoxy, halogen, lower alkyl (substituted or unsubstituted), aryl (substituted or unsubstituted), aralkyl (substituted or unsubstituted), heteroaryl (substituted or unsubstituted), or heteroaralkyl (substituted or unsubstituted);
R₄, independently for each occurrence, represents a substituted or unsubstituted aryl, cycloalkyl, cycloalkenyl, heterocyclyl, heteroaryl, or polycyclyl;
m, independently for each occurrence, represents an integer from 1 to 2; and
n, independently for each occurrence, represents an integer from 0 to 3.

22. A packaged pharmaceutical for a combination therapy, comprising:
a) a direct proteasome inhibitor; and
b) a ubistatin that binds to a K48-linked multiubiquitin chain and inhibits proteasome-based degradation of a polypeptide that is covalently bound to a K48-linked multiubiquitin chain.

23. The composition of claim 22, wherein the ubistatin causes half-maximal inhibition of proteasome-mediated degradation in an in vitro assay at a concentration of about 500 nM or less.

24. The method of claim 23, wherein the ubistatin causes greater than 50% inhibition of the binding of a K48-linked multiubiquitin chain to one or more multiubiquitin binding proteins, such as Rpn10 or Rad23 at concentrations of 5 μM or less.

25. The method of claim 24, wherein the ubistatin binds to a set of amino acid residues on the Ub-Ub interface of the K48-linked chain including at least one amino acid selected from the group consisting of: L8, I44, V70, K6, K11, R42, H68 and R72.

26. The method of claim 24, wherein the compound comprises a purified organic compound having a molecular weight less than 2000 amu, wherein the compound has the Formula (I):
A¹-L¹-B¹-M-B²-L²-A² (I)
or a pharmaceutically acceptable salt or prodrug thereof, wherein as valence and stability permits:
L¹, L², and M independently for each occurrence, represent a direct bond, —(CR¹R²)ₙ—, —(CR¹═CR²)ₙ—, —O—, —S—, —Se—, —NR³—, —(N═N)ₙ—, —O—N═CH—, —(R₃)N—N(R₃)—, —O—N(R³)—, —NR³—C(O)—, —C(O)—NR³—, —C(O)—, —C(S)—, —O—C(O)—O—, —O—C(S)—O—, —NR³—C(O)—NR³—, —O—C(O)—, —C(O)—O—, —NR³—C(S)—NR³—, —O—C(S)—, —C(S)—O—, —S(O)ₘ—, or —C(O)—C(O)—; and
A¹, A², B¹ and B² independently for each occurrence, are absent or represent aryl, heteroaryl, cycloalkyl, cycloalkenyl, heterocyclyl, aralkyl, heteroaralkyl, or polycyclyl, with 1-3 substitutions of R^{a} and 0-3 substitutions of R^{b};
wherein any one of A¹, A², B¹ and B² are optionally connected to any one of the others by one or more bonds;
wherein R^{a}, independently for each occurrence, represents —S(O)ₘ—OH, —CO₂H, —P(O)—(OH)ₘ, —OH, or a moiety having an ionizable hydrogen and a pKa of less than 10;
R^{b}, independently for each occurrence, represents one or more of hydrogen, halogen, hydroxyl, alkoxyl, silyloxyl, amino, nitro, sulfhydryl, alkylthio, imino, amido, cyano, carbonyl, carboxyl, silyl, sulfamoyl, sulfinyl, thioalkyl, alkylsulfonyl, arylsulfonyl, acyl, formyl, esteryl, isocyano, guanidinyl, amidinyl, acetalyl, ketalyl, amine oxidyl, azido, carbamyl, hydroxamyl, imidyl, oximyl, sulfonamidyl, thioamidyl, thiocarbonyl, ureayl, thioureayl, or a substituted or unsubstituted alkyl or, alkenyl, alkynyl, aryl or heteroaryl or —(CH₂)ₙ—R₄;
R¹, R², R³, independently for each occurrence, represent hydrogen, alkoxy, halogen, lower alkyl (substituted or unsubstituted), aryl (substituted or unsubstituted), aralkyl (substituted or unsubstituted), heteroaryl (substituted or unsubstituted), or heteroaralkyl (substituted or unsubstituted);
R₄, independently for each occurrence, represents a substituted or unsubstituted aryl, cycloalkyl, cycloalkenyl, heterocyclyl, heteroaryl, or polycyclyl;
m, independently for each occurrence, represents an integer from 1 to 2; and
n, independently for each occurrence, represents an integer from 0 to 3.
